(19) United States
(12) Patent Application Publication
(10) Pub. No.: US 2022/0072188 A1
(43) Pub. Date: Mar. 10, 2022

(54) Air Sterilizing Apparatus For Lifts

(71) Applicant: Juan Jose Caravaca Hernandez, Murcia (ES)

(72) Inventor: Juan Jose Caravaca Hernandez, Murcia (ES)

(21) Appl. No.: 17/468,283

(22) Filed: Sep. 7, 2021

(30) Foreign Application Priority Data
Sep. 7, 2020 (ES) ES 202031949

(51) Int. Cl. A61L 9/20; A61L 9/014

(52) U.S. Cl. CPC A61L 9/20; A61L 2101/34; A61L 9/014

(57) ABSTRACT

Air Sterilizing Apparatus for Lifts is made up of one or more fans and a series of filters consisting of a HEPA filter, composed of a mesh of fibers, an active carbon filter, designed to eliminate odors, and a titanium filter, which together with the ultraviolet radiation emitted by the germicidal lamp, generates hydroxyl radicals, that allow the sterilization of the air that circulates through the device, purifying the air in the spaces where it is installed.

### PURPOSE OF THE INVENTION

Pursuant to the statement made in this description, this invention is referred to a device getting to sterilize and prevent reproduction from microorganisms such as bacteria, virus, molds, yeast, and protozoa, preserving environment and avoiding fungi and molds. It is specially designed to be placed inside lift and elevator's cabins, though its use in other areas such as hospitals and houses is not ruled out.

Note that microbes such as bacteria, virus, fungi, yeast, and protozoa, whether they are pathogen germs, are vulnerable to ultraviolet light effects at wave lengths next to 253.7 nanometres (range UV-C), the device or equipment subject matter of invention herein acts a sterilizer impacting on molecular structure of microorganisms by removing them and preventing them from reproducing.

Therefore, the air sterilizer device for lifts is supposed to give a proper solution to air sanitization and ethylene removal from lifts and environments susceptible for bacteria and microorganism existence.

Said devices is adding a series of filters which are non-additive preservatives depurating atmospheres and leaving no residue; they aren't toxic or harmful for health, humans or animals. Moreover, it removes odors and volatile organic compounds (VOC) as well as main gases causing odor pollution normally growing in confined spaces such as lifts and elevators.

Technical advantages provided by this air sterilizer device for lifts include the following: Treatment provided by device produces no chemical residue or radiation. Device is effective for sanitization of many surfaces. Equipment is effective to inactivate multiple microorganisms, so handling and application is simple and easy. Device is not supposed to have an expensive manufacturing or maintenance cost. Best temperature range for proper work of said device is −5° C. to 40° C.

Another highlighted feature of device, subject matter of invention herein, is that its whole structure is made of surgical stainless steel 316L. This is chrome nickel austenitic stainless steel containing molybdenum. This addition increases general rust resistance, improves resistance to pitting of chloride ion solutions, and provides a better resistance to high temperature.

In general terms, this invention is referred to an air sterilizer device for lifts, containing one or several fans, which function is to force air flow circulate into filters. Likewise, the device detailed above has a HEPA filter placed which is composed by a fiber mesh, which main function is to catch particles or pathogen elements. It also has an active coal filter, which main function is to remove odors. It has a germicide mechanism in order to remove fungi spores and bacteria present in air flow able to get into the mentioned equipment. Furthermore, it has a Titanium filter which creates hydroxyl radicals along with ultraviolet radiation from germicide lamp allowing sterilization in the air circulating through equipment and purifying are in space where it is mounted.

Therefore, filters mentioned in the above paragraph (HEPA, active coal and titanium) are capable of removing 99.7% of pollutants.

Thus, air sterilizer device for lifts, subject matter of the invention herein, provides as main advantage an optimum air quality in lifts where it is placed. This device is environmentally friendly due to filters added in it, depurating atmosphere without leaving any residue; it is not toxic or harmful for health.

Application field for this invention is the manufacturing industry for air sterilization devices for lifts and other spaces.

### INVENTION BACKGROUND

So far, lifts don't generally have any device or built-in mechanism for air purification. They are confined spaces whereby people frequently move and even pets do sometimes. Due to this, their odors and any kinds of germs create inside lifts since it is necessary to eradicate due to these present circumstances. So, lifts are supposed to be a potentially dangerous spread source.

There are simply ozone devices or humidifiers, but these are used on a domestic or individual basis, not at an industrial level, and they do not have ultraviolet technology as the air sterilizer devices for lifts subject matter of invention herein.

In order to solve matters already stated in the paragraphs above, that is, lifts, and even other confined spaces, have an excellent quality air with no odors, microbes, bacteria or virus, this air sterilizer device for lifts (1), subject matter of invention herein, composed by a container casing (2), one or several funs (3), (4), and (5) which main function is to force air flow circulating into those filters. Likewise, the mentioned device is added with a HEPA filter (6), composed by a fiber mesh, which main function consists of catching particles or pathogen elements. This also has an active coal filter (7), which main function is to remove odors. It has a germicide mechanism with an ultraviolet light (8) so fungi spores and bacteria, located in the air flow, may be removed, and this mechanism can get into the mentioned device. Furthermore, it has a titanium filter (9) which allows air sterilization and creates hydroxyl radicals, along with ultraviolet radiation of the germicide lamp, which allow sterilization of air circulating through the device, purifying air of spaces where it is mounted.

Therefore, the goal of this invention is to provide an air sterilizer device for lifts (1), subject matter of invention herein, which main purpose is to sterilize air circulating through device purifying air in spaces where it is mounted.

### FIGURES DESCRIPTION

In order to complement description herein and make easy to understand invention features, this description is attached to a set of sketches where it is included, but not limited to, illustrations representing the following:

FIG. 1 shows a side view of air sterilizer device for lifts (1), subject matter of invention herein.

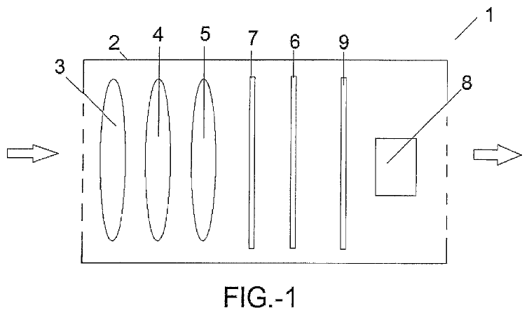

As seen in the referenced figures, an example way of this air sterilizer device for lifts (1), subject matter of invention herein, composed by one or several funs (4), (5), and (6) force air draft getting into the device to different filters, an HEPA filter (6), active coal filter (7), and a titanium filter (9) acting together with the ultraviolet light emitted by germicide lamp (8) in order to remove pathogen elements existing in the space to be sanitized. As an example, any bacterial spore getting into the mentioned device will be sterilized and, once it leaves the device, it will be sterilized with no viral load or completely sterilized with no pollutant capacity.

On the other hand, these manufacturing stages start with the device design and, subsequently, in a second stage, it is needed to proceed with manufacturing of the many parts and elements integrating the invention.

Finally, it proceeds to assembly and installation of the many elements that are part of the device, such as fans, filters, etc., inside the container casing.

As the invention nature has been sufficiently detailed, as well as the way to use it on a practical basis, it is hereby noted that displays previously mentioned and represented in the sketches attached hereto are subjected to be modified in detail provided they do not change the essential purpose.

1.—AIR STERILIZER DEVICE FOR LIFTS (1), featured because it is composed by one or several fans (3), (4), and (5) and a series of filters consisting of a HEPA filter (6). It also has a fiber mesh, an active coal filter (7) targeted to odor removal, and a titanium filter (9) that, along with ultraviolet radiation emitted by germicide lamp (8), creates hydroxyl radicals allowing sterilization of air circulating through device (1) thus purifying air of spaces where it is mounted.

2.—AIR STERILIZER DEVICE FOR LIFTS (1), featured, as per Assertion 1, because it can be installed in any space requiring use frequency of air sterilization.

1. AIR STERILIZING APPARATUS FOR LIFTS (1), characterized in that it is made up of one or more fans, (3), (4) and (5), and a series of filters consisting of a HEPA filter (6), composed of by a mesh of fibers, an active carbon filter (7), designed to eliminate odors, and a titanium filter (9), which together with the ultraviolet radiation emitted by the germicidal lamp (8), generates hydroxyl radicals, that allow the sterilization of the air that circulates through the device (1), purifying the air in the spaces where it is installed.

2. AIR STERILIZING APPARATUS FOR LIFTS (1), characterized, according to claim 1, because it can be installed in any space that requires, due to its frequency of use, the sterilization of the air.
